(19) United States
(12) Patent Application Publication
(10) Pub. No.: US 2010/0331257 A1
(43) Pub. Date: Dec. 30, 2010

(54) PREPARATION OF TERMINALLY-STERILIZED COLLAGEN THAT IS SOLUBLE AT NEUTRAL PH

(75) Inventors: Yves Bayon, Lyon (FR); Philippe Gravagna, Irigny (FR)

Correspondence Address: CARTER, DELUCA, FARRELL & SCHMIDT, LLP, 445 BROAD HOLLOW ROAD, SUITE 420, MELVILLE, NY, 11747, US

(73) Assignee: SOFRADIM PRODUCTION, Trevoux (FR)

(21) Appl. No.: 12/865,039

(22) PCT Filed: Jan. 30, 2009
(86) PCT No.: PCT/IB09/00325

§ 371 Date: Jul. 28, 2010

Related U.S. Application Data
(60) Provisional application No. 61/025,027, filed on Jan. 31, 2008.

(51) Int. Cl. A61K 38/39; C07K 14/78; A61P 19/08; A61P 17/02; A61L 2/08

(52) U.S. Cl. 514/17.2; 530/356; 422/22

(57) ABSTRACT

Terminally sterilized collagen that is soluble at a neutral pH is useful for medical uses. The invention also relates to the method for preparing such terminally sterilized collagen.

### BACKGROUND

The present disclosure relates to collagen compositions and methods for their preparation. More specifically, terminally sterilized collagen which remains fully soluble at a neutral pH while maintaining most of the initial properties of native collagen, like in particular viscosity characteristics, is described.

In the present disclosure, when the term “soluble” is used with regards to collagen, it is meant that the collagen is soluble in water.

Techniques for the preparation and sterilization of collagen are known. In some cases, sterilization is performed before the final steps of the collagen preparation process. It is difficult, however, in such cases to maintain strictly sterile conditions during the final steps of the preparation process.

Difficulties also arise in collagen preparation processes that employ terminal sterilization of collagen. Often such processes do not produce collagen that is fully soluble at a neutral pH while retaining the physiological properties, potency and effectiveness of native collagen. Rather than maintaining the properties of native collagen, previously disclosed terminally sterilized collagen compositions can be crosslinked, denatured and/or not fully soluble at a neutral pH.

Yet, it would be interesting to have at hand terminally sterilized collagen that is fully soluble at a neutral pH while retaining the characteristics of native collagen, for example for use as component of an adhesive, for which the solubility of the collagen may be important. Moreover, it would also be interesting to have terminally sterilized collagen that is fully soluble at a neutral pH while retaining the characteristics of native collagen, so as to allow the preparation of solutions of sterilized collagen having a viscosity enabling the use of such solutions as injectable products which can be used for example for the treatment of wrinkles and urinary incontinence, for filling defects (eg. bone defects).

It would be advantageous to provide a process leading to terminally sterilized collagen that is fully soluble at a neutral pH while retaining the characteristics of native collagen.

### SUMMARY

Methods for making collagen preparations include chemically modifying collagen to render the collagen soluble at physiological pH, neutralizing the chemically modified collagen to a physiological pH, optionally drying the neutralized chemically modified collagen, packaging the neutralized chemically modified collagen in sealed units, and irradiating the packaged, neutralized chemically modified collagen with a dose of radiation, for example beta-radiation, equal to or lower than about 25 KGy. In embodiments, the collagen is chemically modified by reacting collagen with a water soluble aliphatic alcohol to esterify the collagen.

In embodiments, methods for making collagen preparations include chemically modifying collagen to render the collagen soluble at physiological pH, neutralizing the chemically modified collagen to a physiological pH, optionally drying the neutralized chemically modified collagen, packaging the neutralized chemically modified collagen, and irradiating the packaged, neutralized chemically modified collagen with a dose of radiation, for example beta-radiation, equal to or lower than about 25 KGy.

In embodiments, the dried, neutralized chemically modified collagen is stored at or below about 30° C. before the irradiating step.

In embodiments, the irradiating is irradiating with beta-radiation, for example at a dosage from about 6 KGy to about 25 KGy, preferably at a dosage from about 6 KGy to about 15 KGy, and more preferably at a dosage from about 10 KGy to about 15 KGy. In embodiments, the irradiating with beta-radiation is at a dosage lower than about 15 KGy, preferably lower than about 10 KGy.

In embodiments, irradiating is performed as multiple cycles of irradiating doses wherein the total dose of radiation is equal to or lower than about 25 KGy.

In embodiments, irradiating is performed at a temperature below 0° C.

In embodiments, chemically modifying collagen comprises esterifying collagen, for example by reacting collagen with a water soluble aliphatic alcohol.

In embodiments, packaging comprises sealing the neutralized chemically modified collagen in a syringe.

Another aspect of the present disclosure is a collagen preparation comprising terminally sterilized neutralized chemically modified collagen that is soluble at physiological pH. In embodiments, the collagen has at least in part an intact triple helical structure. In embodiments, the collagen is porcine.

Another aspect of the present disclosure is a fiber formed from a collagen preparation as described above. Another aspect of the present disclosure is a tissue sealant or tissue adhesive comprising a collagen preparation as described above. The tissue sealant or tissue adhesive may further include oxidized starch.

Another aspect of the present disclosure is a collagen preparation as described above in combination with a bioactive agent.

Another aspect of the present disclosure is a collagen preparation as described above that is injectable and contained within a syringe.

Another aspect of the present disclosure is a bone filler comprising a collagen preparation as described above that contains one or more bone morphogenic proteins, is injectable and contained within a syringe.

Another aspect of the present disclosure is a method comprising:

combining a collagen preparation comprising terminally sterilized neutralized chemically modified collagen that is soluble at physiological pH with a sterile composition comprising a bioactive agent to provide a formulation suitable for application to tissue. The present disclosure relates to formulation comprising i) a collagen preparation comprising terminally sterilized neutralized chemically modified collagen that is soluble at physiological pH and ii) a sterile composition comprising a bioactive agent.

In embodiments, the collagen preparations described herein are formulated to be injectable and may be packaged in syringes. In embodiments, the collagen preparations described herein are formulated to be suitable for use as a tissue sealant and a tissue adhesive. In embodiments, the collagen preparations described herein are formulated to include one or more bone morphogenic proteins to render them suitable for repair of bone or cartilage defects.

### BRIEF DESCRIPTION OF THE DRAWINGS

FIG. 1A shows the DSC (differential scanning calorimetry) profile of non-β-irradiated acid collagen as a control.

FIG. 1B shows the DSC profile of acid collagen that has been β-irradiated at 6 KGy.

FIG. 1C shows the DSC profile of acid collagen that has been β-irradiated at 10 KGy.

FIG. 2A shows the DSC profile of non-β-irradiated esterified collagen as a control.

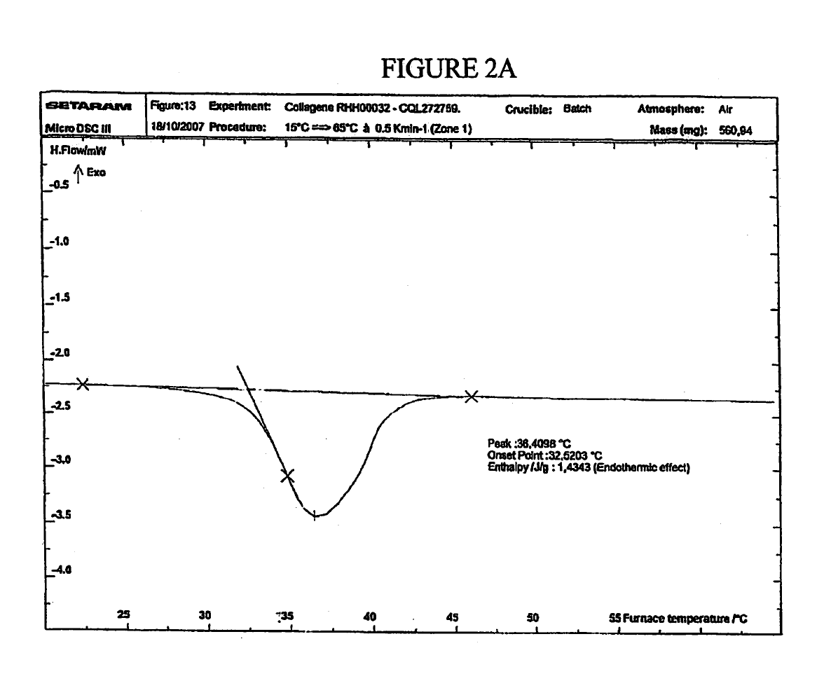

FIG. 2B shows the DSC profile of esterified collagen that has been β-irradiated at 6 KGy.

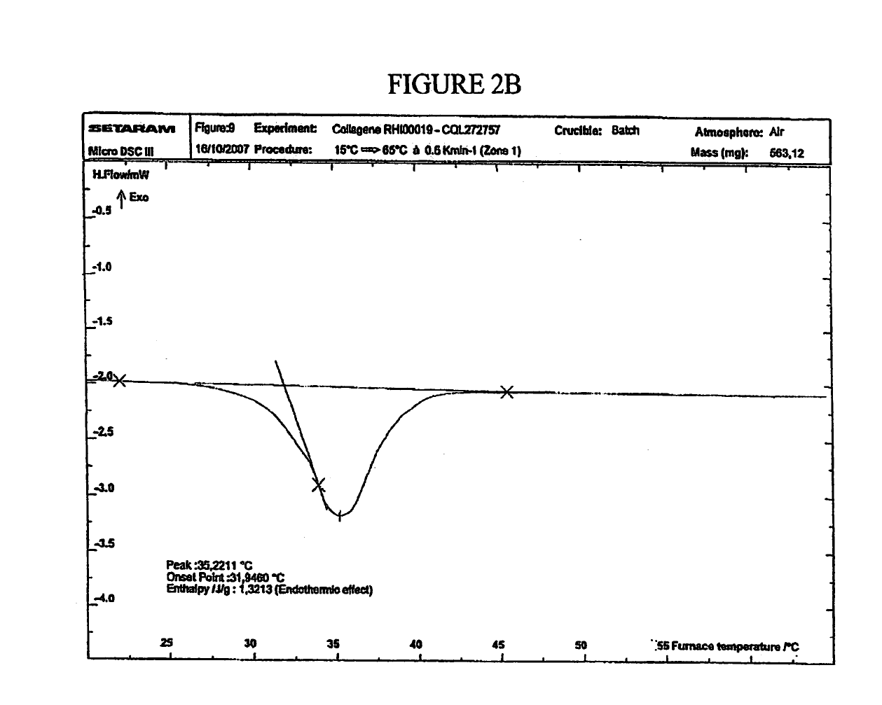

FIG. 2C shows the DSC profile of esterified collagen that has been β-irradiated at 10 KGy.

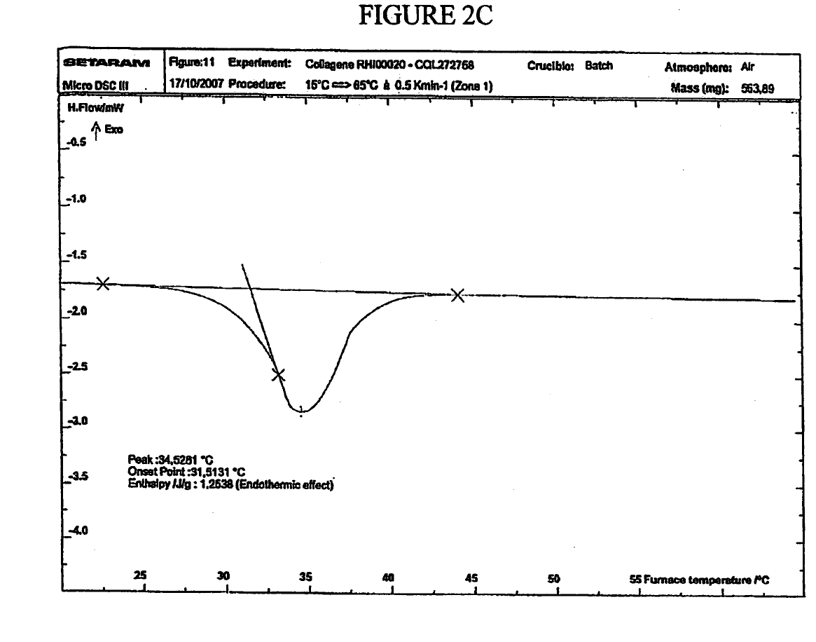

FIG. 3A shows the DSC profile of non-β-irradiated neutral collagen as a control.

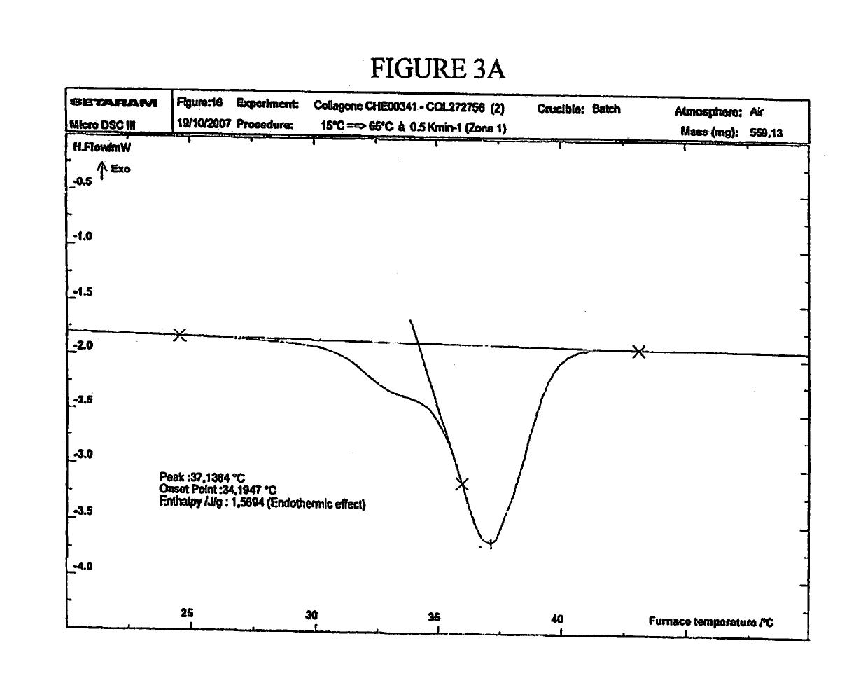

FIG. 3B shows the DSC profile neutral collagen that has been β-irradiated at 6 KGy;

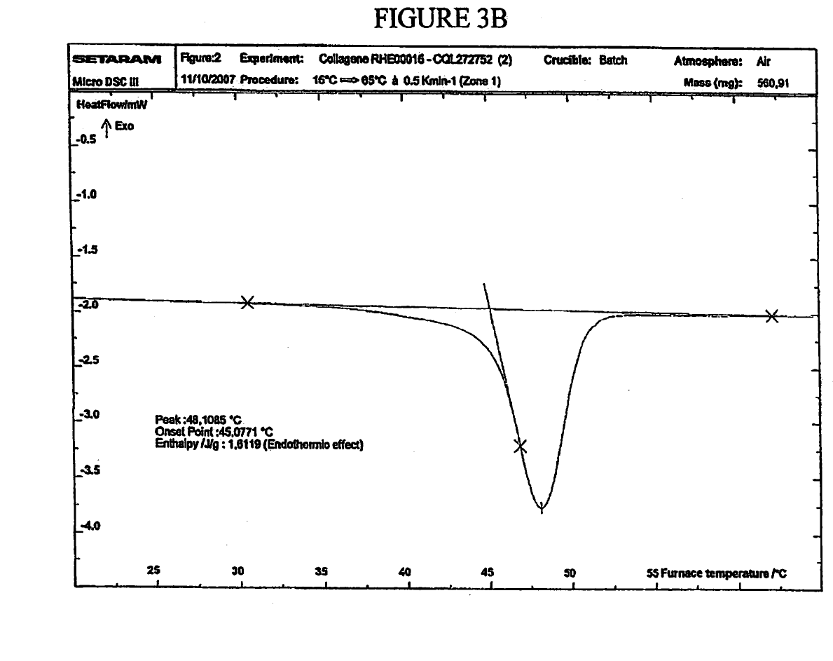

FIG. 3C shows the DSC profile neutral collagen that has been β-irradiated at 10 KGy.

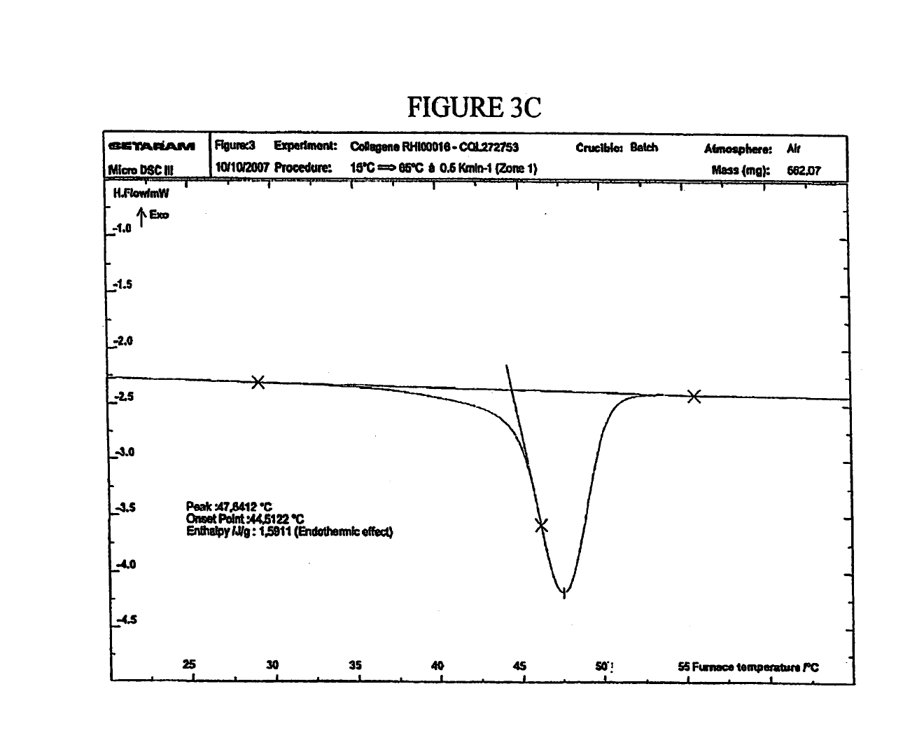

FIG. 4 shows the electrophoretic pattern of β-irradiated collagens, specifically: β-irradiated neutral collagen (samples RHE00015 β-irradiated at 6 KGy and RHI00016 β-irradiated at 10 KGy); β-irradiated acid collagen (samples RHI00017 β-irradiated at 6 KGy and RHI00018 β-irradiated at 10 KGy); β-irradiated esterified collagen (samples RHI00019 β-irradiated at 6 KGy RHI00020 β-irradiated at 10 KGy); non-β-irradiated collagen (control sample CHE00341); and non-β-irradiated esterified collagen (control sample RHH00032).

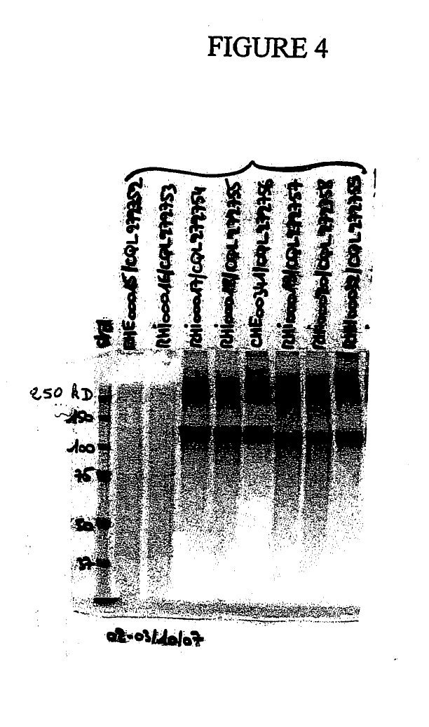

FIG. 5 shows the electrophoretic pattern of γ-irradiated collagens, specifically: γ-irradiated esterified collagen (samples RGI00008 γ-irradiated at 8-9 KGy, RGI00009 γ-irradiated at 6-8 KGy in dry ice, RGI00010 γ-irradiated at 14-15 KGy, RGI00011 γ-irradiated at 11-14 KGy in dry ice, RGI00012 γ-irradiated at 29-34 KGy, and RGI00013 γ-irradiated at 28-30 KGy in dry ice).

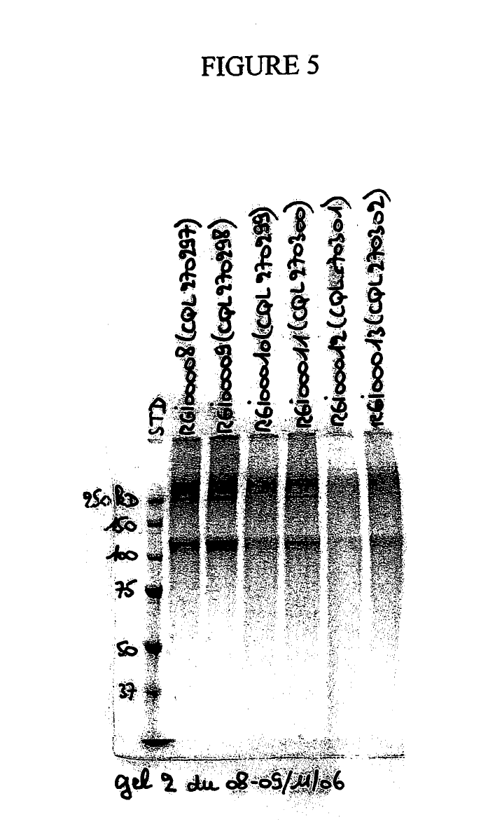

### DETAILED DESCRIPTION

Collagen preparations in accordance with the present disclosure are for example prepared by exposing chemically modified collagen to a dose of beta-irradiation sufficient to effect sterilization. The collagen material is first chemically modified, so as to obtain collagen which is soluble at a physiologic pH and at body temperature. It is then neutralized at a physiological pH and optionally dried before being terminally sterilized. The processes for preparing and irradiating collagen are described in greater detail below.

Collagen of any type may be used, including, but not limited to types I, II, III, IV, and V, forms of minor collagen, or any combinations thereof. The collagen may be from any source, such as, for example, human, bovine, or porcine, and may be derived from the skin, tendon, muscle, connective tissues, or any other naturally occurring structural element having a high collagen content. Collagen may also be synthetic collagens recombinantly or otherwise produced, such as, for example, by tissue engineering. In embodiments, type I porcine or bovine collagen is used. In other embodiments, type I and type III collagen from different animal origins, such as bovine, porcine, human, or mixtures in any proportions of types are used.

Collagen refers to all forms of collagen, including those which have been processed or otherwise modified. Native collagen is characterized as fibrous proteins that are long, rigid rod-like structures that are made up of three polypeptide chains wound together in a triple helix configuration having non-helical terminal portions. In embodiments, the collagen used has intact triple helical structures to provide collagen preparations which display a much higher viscosity in solution and that are less prone to degradation by enzymes than thermally denatured collagens. In embodiments, thermally denatured collagens which have at least partially lost its helical structure, commonly known as gelatins, may be used.

Atelopeptide or telopeptide-containing collagen may be used. Telopeptides, the nonhelical terminal portions of collagen, extend as random coils from the amino and carboxy ends of the collagen molecules. The telopeptides serve as the primary sites for intramolecular and intermolecular cross-linking and are sites of immunogenicity. In embodiments, atelocollagens from an animal source, such as a bovine or porcine source, are used because of their reduced immunogenicity.

In order to minimize immunogenicity of the collagen used, the telopeptides can be removed. Atelocollagens are produced by any technique known to those skilled in the art, including, but not limited to, methods using proteolytic enzymes. For example, pepsin may be used as described by Miyata, et al. in U.S. Pat. No 4,164,559, the entire contents of which are incorporated herein by this reference. Briefly, this technique involves solubilizing a collagen material with pepsin and precipitating the soluble collagen at a pH of about 7.0 after in-activation of enzyme activity by caustic treatment at a pH of about 10.0. The collagen is purified by repeating re-dissolution in acidic water at a pH of about 2.0 to about 4.0 and re-precipitation at a pH of about 7.0.

For example, to prepare soluble collagen at a physiological pH, a collagen material or source, such as skin or tendon, is cleaned to remove non-collagen components such as hair, fat, carbohydrates, mucopolysaccharides, and the like. The collagen material is then solubilized. Soluble collagen at a physiological pH refers to collagen that is chemically modified in such a way that it is soluble at a physiological pH and at body temperature by any known methods, such as by acid extraction or with a proteolytic enzyme, such as pepsin, as described above. Many proteolytic enzymes which are not specific collagenases may be used to solubilize collagen without denaturation. For example, in addition to pepsin, trypsin, chymotrypsin, or papain may be used.

Chemical modification of collagen to provide soluble collagen at a physiological pH may be accomplished by esterification of the collagen carboxyl groups by any known methods, including reaction with acidified alcohol, such as a water-soluble aliphatic alcohol such as, methanol, ethanol, and the like. Suitable techniques for chemically modifying collagen to render the collagen soluble at physiological pH are described, for example in U.S. Pat. No. 4,164,559, the entire contents of which are incorporated herein by this reference. For example, ethylation of type I atelocollagen renders them soluble at a pH of about 5.5 to about 9.0 and, particularly, in the range of 7.0 to 7.5. The unmodified atelocollagen is soluble only in an acidic pH lower than 5.0 or in an alkaline pH above 9.0.

Once collagen, soluble at a physiological pH, is obtained, it is for example neutralized at a pH from about 6.0 to about 8.0, in embodiments at a physiological pH. Physiological pH refers to a pH that is in a relatively narrow range as normally encountered in the fluids of the human body, generally in the range of about 7.0 to about 7.5.

The soluble collagen at a physiological pH is then optionally dried by any known relevant methods, including, but not limited to, drying in an oven under vacuum, freeze drying, and dehydration by solvents such as acetone.

The optionally dried collagen is packaged under any suitable known sealed or closed units relevant to the final use of such collagens. Packaging units include syringes, vials, pouches, plates, jars, tubes, and any other casing appropriate for the ultimate use of product. The terminally packaged collagen may then be stored at a temperature below 30° C. until it is sterilized by beta-irradiation.

Sterilization is generally performed on the terminally packaged collagen by irradiation. Sterilization refers to collagen that is treated via a single procedure or a combination of procedures which reduce the number of microorganisms capable of growing in the collagen under conditions at which the collagen is stored and/or distributed and is below a level determined by a standardized sterilization protocol and/or validation test. Terminal sterilization refers to collagen that is sterilized as defined above, in its final packaging for storage and distribution prior to use.

The sterilization process is for example performed at room temperature or any temperature lower than room temperature according to any known suitable methods. Room temperature refers to the temperature generally recorded in irradiation facilities, in embodiments, room temperature is less than 50° C., more generally 25° C.±5° C. In embodiments, sterilization is effected on dry ice at a temperature below 0° C.

Sterilization is performed at a sterilizing total dose equal to or lower than 25 KGy. In embodiments, the sterilization is performed by beta-irradiation at a sterilizing total dose lower than 15 KGy. In embodiments, the sterilization is performed by beta-irradiation at a sterilizing total dose lower than 10 KGy. Sterilizing doses may include one or more cycles of irradiation except that the total cumulative dose should by lower than or equal to values mentioned above.

Collagen soluble at physiological pH, particularly esterified collagen, is not extensively degraded by the beta-irradiation sterilization treatment. The collagen keeps most of its initial properties (e.g. molecular weight profile, viscosity, thermal properties, etc). For example, ethylated type I atelocollagen remains soluble at a physiological pH after its sterilization by beta-irradiation. A solution of ethylated type I atelocollagen is as viscous as it is before beta-irradiation. By contrast type I collagen is degraded to such an extent that it is no longer soluble.

Use of beta-irradiated esterified collagen can be unlimitedly envisaged wherever specialists in relevant fields might advantageously consider using terminally sterilized collagens with features similar to native collagen. Non-limiting examples of how beta-irradiated esterified collagen may be used include, but are not limited to, as a component of sealants, adhesives, matrices for healing and regeneration of tissues and organs, wound dressings, osteogenic formulations, chondrogenic formulations, matrices for drug delivery, hemostatic products, cosmetic formulations, fillers for wrinkles and lips, skin creams, as well as, a medium for cell cultures, medium for microorganism cultures, and a reagent for in vitro and in vivo tests.

In embodiments, beta-irradiated esterified collagen can be used in the formation of fibers and devices made from such fibers. Matrices, scaffolds, and meshes made from the collagen of the present disclosure and produced in accordance with the methods described herein have properties particularly useful in medical and surgical applications. For example, applications include, but are not limited to, surgical sutures, blood vessel grafts, catheters, and in general, the fabrication of surgical prostheses and artificial organs. Techniques for making fibers from collagen are within the purview of those skilled in the art and include, but are not limited to the techniques disclosed in U.S. Pat. Nos. 6,997,231 and 6,361,551 the entire disclosures of which are incorporated herein by this reference.

Beta-irradiated esterified collagen, after some handling and just before use, can be used in the form of pastes, gels, solutions, or suspensions, homogeneous or heterogeneous, which are contained in syringes, tubes, or other containers equipped with appropriate plungers, sprayers, or systems designed to extrude the collagen content, such as through a needle or a nozzle. The collagen may be injected, surgically applied through a trocar, or directly applied on a wound surface. The collagen paste, gel, solution, or suspension is of a similar viscosity before and after its terminal sterilization as it remains fully soluble.

In embodiments, at least one bioactive agent may be combined with the collagen for use in the present compositions. In these embodiments, the collagen preparation can also serve as a vehicle for delivery of the bioactive agent. The term “bioactive agent”, as used herein, is used in its broadest sense and includes any substance or mixture of substances that have clinical use. Consequently, bioactive agents may or may not have pharmacological activity per se, e.g., a dye, or fragrance. Alternatively a bioactive agent could be any agent which provides a therapeutic or prophylactic effect, a compound that affects or participates in tissue growth, cell growth, cell differentiation, an anti-adhesive compound, a compound that may be able to invoke a biological action such as an immune response, or could play any other role in one or more biological processes.

In embodiments, the bioactive agent is added to irradiated, esterified collagen immediately prior to use. In such embodiments, the bioactive agent can be separately provided in a sterile package of known types, such as within a syringe. As one example, the contents of a syringe of sterile collagen can be mixed with the contents of a syringe containing sterile bone morphogenic protein (BMP) to provide a formulation suitable for bone repair.

In other embodiments, one or more bioactive agents are added to the collagen preparation prior to sterilization of the collagen.

Examples of classes of bioactive agents which may be utilized in accordance with the present disclosure include anti-adhesives, antimicrobials, analgesics, antipyretics, anesthetics, antiepileptics, antihistamines, anti-inflammatories, cardiovascular drugs, diagnostic agents, sympathomimetics, cholinomimetics, antimuscarinics, antispasmodics, hormones, growth factors, muscle relaxants, adrenergic neuron blockers, antineoplastics, immunogenic agents, immunosuppressants, gastrointestinal drugs, diuretics, steroids, lipids, lipopolysaccharides, polysaccharides, and enzymes. It is also intended that combinations of bioactive agents may be used.

Anti-adhesive agents can be used to prevent adhesions from forming between implantable medical devices and the surrounding tissues opposite the target tissue. Some examples of these agents that may be included in the present compositions include, but are not limited to poly(vinyl pyrrolidone), carboxymethyl cellulose, hyaluronic acid, polyethylene oxide, poly vinyl alcohols and combinations thereof.

Suitable antimicrobial agents which may be included as a bioactive agent in the compositions of the present disclosure include triclosan, also known as 2,4,4′-trichloro-2′-hydroxydiphenyl ether, chlorhexidine and its salts, including chlorhexidine acetate, chlorhexidine gluconate, chlorhexidine hydrochloride, and chlorhexidine sulfate, silver and its salts, including silver acetate, silver benzoate, silver carbonate, silver citrate, silver iodate, silver iodide, silver lactate, silver laurate, silver nitrate, silver oxide, silver palmitate, silver protein, and silver sulfadiazine, polymyxin, tetracycline, aminoglycosides, such as tobramycin and gentamicin, rifampicin, bacitracin, neomycin, chloramphenicol, miconazole, quinolones such as oxolinic acid, norfloxacin, nalidixic acid, pefloxacin, enoxacin and ciprofloxacin, penicillins such as oxacillin and pipracil, nonoxynol 9, fusidic acid, cephalosporins, and combinations thereof. In addition, antimicrobial proteins and peptides such as bovine lactoferrin and lactoferricin B and antimicrobial polysaccharides such as fucans and derivatives may be included as a bioactive agent in the bioactive coating of the present disclosure.

Other bioactive agents which may be included as a bioactive agent in the compositions in accordance with the present disclosure include: local anesthetics; non-steroidal antifertility agents; parasympathomimetic agents; psychotherapeutic agents; tranquilizers; decongestants; sedative hypnotics; steroids; sulfonamides; sympathomimetic agents; vaccines; vitamins; antimalarials; anti-migraine agents; anti-parkinson agents such as L-dopa; anti-spasmodics; anticholinergic agents (e.g. oxybutynin); antitussives; bronchodilators; cardiovascular agents such as coronary vasodilators and nitroglycerin; alkaloids; analgesics; narcotics such as codeine, dihydrocodeinone, meperidine, morphine and the like; non-narcotics such as salicylates, aspirin, acetaminophen, d-propoxyphene and the like; opioid receptor antagonists, such as naltrexone and naloxone; anti-cancer agents; anti-convulsants; anti-emetics; antihistamines; anti-inflammatory agents such as hormonal agents, hydrocortisone, prednisolone, prednisone, non-hormonal agents, allopurinol, indomethacin, phenylbutazone and the like; prostaglandins and cytotoxic drugs; estrogens; antibacterials; antibiotics; anti-fungals; anti-virals; anticoagulants; anticonvulsants; antidepressants; antihistamines; and immunological agents.

Other examples of suitable bioactive agents which may be included in the present compositions include viruses and cells, peptides, polypeptides and proteins, analogs, muteins, and active fragments thereof, such as immunoglobulins, antibodies, cytokines (e.g. lymphokines, monokines, chemokines), blood clotting factors, hemopoietic factors, interleukins (IL-2, IL-3, IL-4, IL-6), interferons ((3-IFN, (a-IFN and γ-IFN), erythropoietin, nucleases, tumor necrosis factor, colony stimulating factors (e.g., GCSF, GM-CSF, MCSF), insulin, anti-tumor agents and tumor suppressors, blood proteins, gonadotropins (e.g., FSH, LH, CG, etc.), hormones and hormone analogs (e.g., growth hormone), vaccines (e.g., tumoral, bacterial and viral antigens); somatostatin; antigens; blood coagulation factors; growth factors (e.g., nerve growth factor, insulin-like growth factor); factors useful in the repair of bone or cartilage such as bone morphogenic proteins (BMPs); protein inhibitors, protein antagonists, and protein agonists; nucleic acids, such as antisense molecules, DNA and RNA; oligonucleotides; polynucleotides; and ribozymes.

The following non-limiting examples show properties of beta-irradiated esterified collagen as well as a comparison data of the beta-irradiated esterified collagen with the beta-irradiated acid and neutral collagens, as well as with gamma-irradiated esterified collagen.

### Example 1

### Preparation of Collagen

Type I porcine collagen is extracted from porcine dermis and rendered soluble by various techniques to produce acid collagen, neutral collagen, and esterified collagen:

### 1°) Acid Collagen

Type I porcine collagen is solubilized at an acidic pH or by pepsin digestion and then purified by saline precipitation using conventional techniques. Dry collagen fibers are obtained by the precipitation of an acid solution of collagen by adding NaCl, and then washing and drying the precipitate obtained with an aqueous solution of acetone having an increasing concentration from 80% to 100%.

### 2°) Neutral Collagen

Type I porcine collagen obtained as described above at 1°) is solubilized in water at a concentration of 30 g/l. The pH of the subsequent collagen preparation is neutralized with the addition of dilute sodium hydroxide solution, at a pH between 7.0 and 7.5. The collagen is then dried either by acetone washing or by freeze-drying.

### 3°) Esterified Collagen

Twenty grams of dry collagen fibers as obtained at 1°) above are immersed in one liter of dehydrated ethanol containing 0.1 N HCl for seven days at room temperature in a closed vessel. Dehydration of ethanol containing HCl prior to the addition of collagen is carried out by intermittent stirring with excess of anhydrous sodium sulphate. After ethylation, the solvent is removed.

The esterified collagen fibers are then washed several times with acetone. They are then dried and solubilized in water at a final concentration of 1% w/v. The pH of the solution is adjusted between 7.0 and 7.5 and the collagen is freeze-dried.

### Sterilization of Collagen

The acid collagen, neutral collagen, and esterified collagen obtained as described above are individually filled in syringes. The syringes are then packed in sealed bags. The collagen syringes are sterilized by beta-irradiation at different doses ranging from 6 KGy to 25 KGy.

The esterified collagen as described above is also sterilized by beta-irradiation at different doses, ranging from 6 KGy to 25 KGy, on dry ice, at a temperature lower than 0° C.

### Analysis of Beta-irradiated Collagens

Properties of the beta-irradiated collagens are shown individually for beta-irradiated esterified collagen and comparatively for beta-irradiated acid collagen, neutral collagen, and esterified collagen.

### Sterility of Beta-irradiated Esterified Collagens

Sterility tests were performed on different batches of irradiated esterified collagen prepared as described above, according to the European Pharmacopoeia sterility standards. All tested batches were found sterile as shown in Table 1:

TABLE 1

| Batch: | Beta-irradiation: | Results: |
|---|---|---|
| RGI00009 | 6 KGy | Sterile |
| RGI00010 | 6 KGy + dry ice during irradiation | Sterile |
| RGK00026 | 6 KGy | Sterile |
| RHE00021 | 6 KGy | Sterile |
| RHI00019 | 6 KGy | Sterile |
| RGI00011 | 10 KGy | Sterile |
| RGI00012 | 10 KGy + dry ice during irradiation | Sterile |
| RHE00022 | 10 KGy | Sterile |
| RHE00020 | 10 KGy | Sterile |
| RGI00013 | 25 KGy | Sterile |
| RGI00014 | 25 KGy + dry ice during irradiation | Sterile |

### Solubility and Viscosity of Beta-irradiated Esterified Collagen

The solubility of Beta-irridated esterified collagen was evaluated as follows: Collagen was first solubilized by incorporating 150 mg of dry collagen (assumed residual water content: 10%) in 100 ml of water, at a neutral pH. The preparation was centrifuged at 10 000 rpm, during 4 min. The total amount of collagen in the supernatant was dosed by the Biuret method and was compared to the total amount of collagen initially present in the preparation, before its centrifugation. As shown in Table 2, irradiated esterified collagens were as soluble as non-irradiated esterified collagens.

TABLE 2
Solubility

| Batch of esterified collagen | Beta-irradiation | Solubility |
|---|---|---|
| RIA00027 | None | >95% |
| RIA00028 | 6 KGy | >95% |
| RIA00029 | 10 KGy | >95% |

The plastic viscosity and yield stress of different batches of beta-irradiated esterified collagen were measured at +25° C., in solution, at a concentration of 2% w/v, according to Bingham's model. Values were acquired with the TVe-05 Lamy viscosimeter, between 300 and 600 rpm. As shown in Table 3, values were in the same range before and after beta-irradiation, at 6 KGy and 10 KGy. Irradiated esterified collagen remained fully soluble when beta-irradiated at 6KGy and 10 KGy, and only partially soluble at 25 KGy.

TABLE 3

| | | Plastic Viscosity | Yield Stress |
|---|---|---|---|
| Batch: | Beta-irradiation: | (mPa · s) | (Pa · s) |
| RHE00012 | None | 62 | 140 |
| RHE00021* | 6 KGy | 60 | 141 |
| RHE00022* | 10 KGy | 71 | 135 |

*RHE00021 and RHE00022 batches were obtained by irradiation of RHE00012 batch of esterified collagen

### Compared Solubility and Viscosity of Beta-irradiated Collagens

The viscosity of the different batches of beta-irradiated esterified collagen and beta-irradiated acid collagen were measured at +25° C., in solution, at a concentration of 2% w/v, as described above. Viscosity measurements were also carried out on neutral collagen, after heating to a temperature of +50° C., so as to obtain gelatin.

Beta-irradiated neutral collagen was not soluble, whatever the dose of beta-irradiation, either at an acid pH or after heating at +42° C. at a neutral pH. After heating, the solution did not form a gel at room temperature, even at a concentration up to 4% w/v whereas non-irradiated neutral collagen, after heating, gave a gel at a concentration lower than 1% w/v at room temperature. Irradiated acid collagen was soluble, but did not gelify after heating at +42° C. Irradiated esterified collagen was as soluble as the esterified collagen that had not been irradiated.

TABLE 4

| | Beta- | Plastic Viscosity | Yield Stress |
|---|---|---|---|
| Batch: | irradiation: | (mPa · s) | (Pa · s) |
| Esterified Collagen | | | |
| RHE00012 | None | 62 | 140 |
| RHI00019 | 6 KGy | 62 | 132 |
| RHI00020 | 10 KGy | 56 | 123 |
| Neutral Collagen | | | |
| RHC00046 (0.3%*) | None | 11 | 18 |
| RHC00046 (1.0%*) | None | na** | na** |
| RHI00015 | None | 12 | 17 |
| Acid Collagen | | | |
| CHG00025 | None | 51 | 89 |
| RHI00017 | 6 KGy | 52 | 131 |
| RHI00018 | 10 KGy | 63 | 135 |

*concentration of collagen (w/v)
**viscosity not measured since at this concentration, neutral collagen is already a gel, at room temperature

### Thermal Properties of Beta-Irradiated Collagens

Thermal properties were measured by differential scanning calorimetry (DSC) with a Micro-DSC III device (Tian-Calvet Type) at +23° C. from 15° C. to 65° C. at a speed of 0.5 K/min. Readings were obtained from wet samples of collagen fibers, prepared by swelling 15 mg of collagen fibers in 500 μl of de-mineralized water.

Thermal properties were not substantially modified for esterified collagen after beta-irradiation up to 10 KGy, whereas they were degraded for irradiated acid and neutral collagens, degraded to a larger extent for the latter.

The results shown in the following Table 5, shows more clearly the alteration of thermal properties by beta-irradiation.

TABLE 5

| | | | Denaturation |
|---|---|---|---|
| | Beta- | Denaturation ΔH | Temperature |
| Batch: | irradiation: | (J/g dry collagen) | (° C.) |
| Esterified Collagen | | | |
| RHH00032 | None | 51 | 36 |
| RHI00019 | 6 KGy | 50 | 35 |
| RHI00020 | 10 KGy | 48 | 35 |
| Neutral Collagen | | | |
| CHE00341 | None | 60 | 37 |
| RHI00015 | 6 KGy | 63 | 48 |
| RHI00016 | 10 KGy | 61 | 48 |
| Acid Collagen | | | |
| CHE00341 | None | 60 | 37 |
| RHI00017 | 6 KGy | 56 | 33/37* |
| RHI00018 | 10 KGy | 50 | 33/37* |

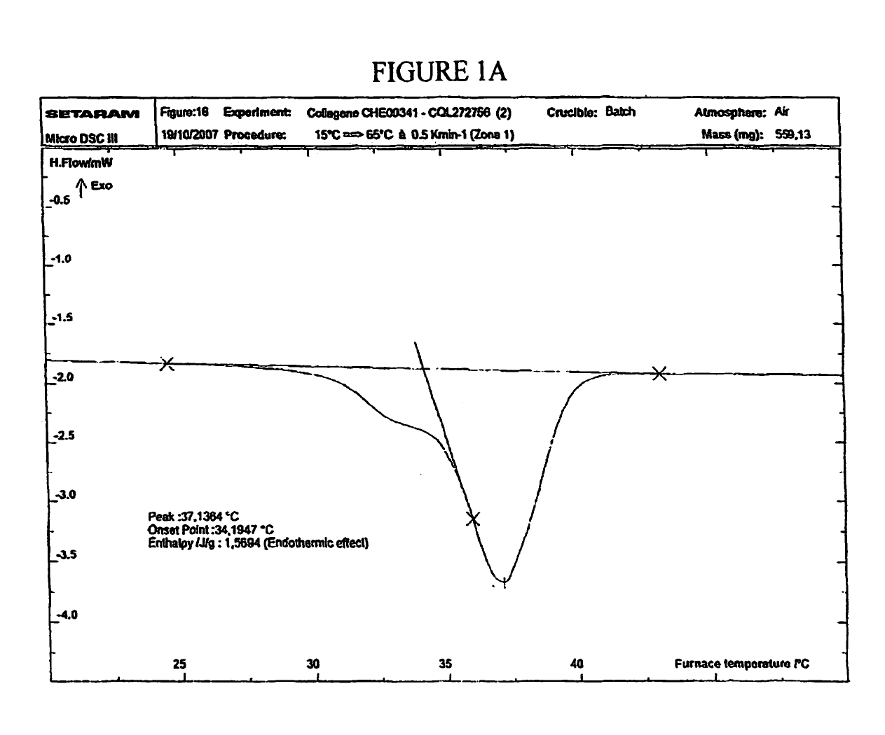

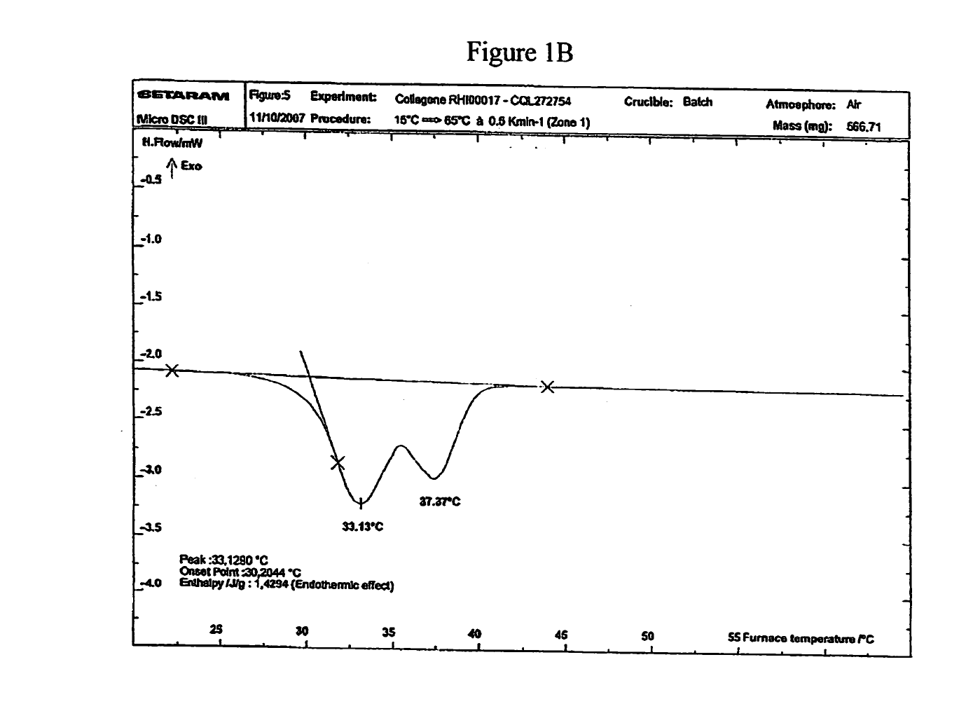

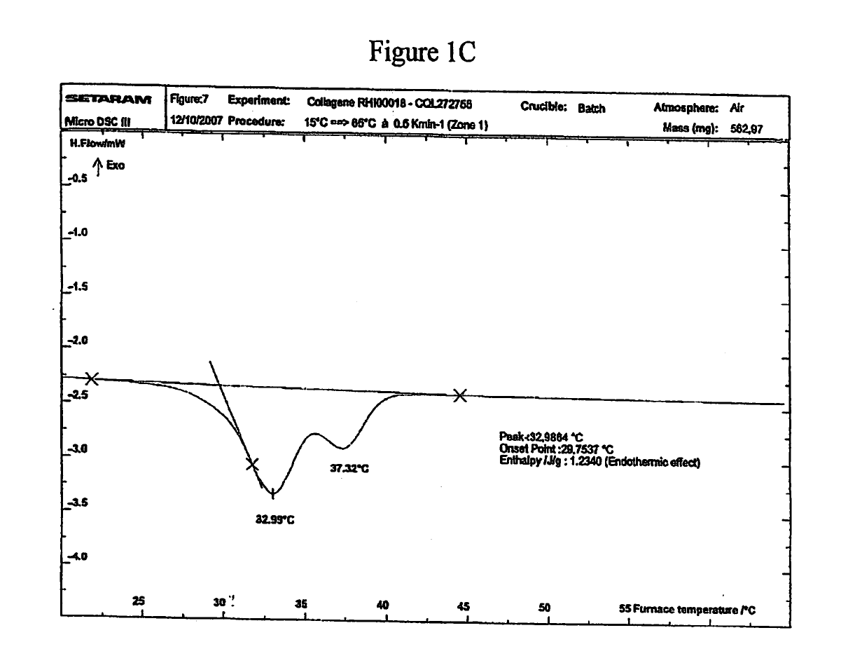

*the denaturation peak was clearly split in two (see FIGS. 1B and 1C)

Electrophoretic Properties of Beta-irradiated Collagens Electrophoresis of collagen was carried out on Criterion™ XT 7% Tris Acetate precast gels, by using the running buffer XT Tricine Running Buffer 20× (BioRad). Sample preparation included XT Reducing Agent (BioRad). Gels were stained with Bio-Safe Coomassie (BioRad), according to BioRad instructions.

Beta-irradiated neutral collagen showed a deeply altered electrophoretic profile, with no visible bands, but with an extended trail. Electrophoretic properties of esterified collagen and acid collagen were not substantially modified after beta-irradiation, at doses up to 10 KGy.

### Functional Test: Adhesive Strength of Beta-irradiated Esterified Collagen Based Formulations

Adhesive collagen based compositions for surgical and/or therapeutic use, especially for the bonding of biological tissues to one another or to an implanted biomaterial, may be prepared as described by Tardy et al. in U.S. Pat. No. 6,165,488, the entire contents of which are incorporated herein by this reference. Briefly, the composition generally contains an aqueous solution of oxidized starch, a biodegradable polysaccharide, and an aqueous solution of heated collagen (i.e. gelatine).

The composition is obtained by mixing together oxidized starch and collagen, by incorporating air bubbles, or not, at a neutral pH. The mixing provides a gel which is rapidly applied to tissues and/or biomaterials to achieve binding. The adhesiveness is the result of the combination of the chemical reaction of oxidized starch aldehydes with the free amino groups of collagen as well as the unique properties of collagen (e.g., viscosity or hydropathic properties).

For the functional test, the heated collagen was replaced with esterified collagen beta-irradiated at a dose of 6 KGy.

Fully translucent solutions of irradiated collagen have been obtained, at concentrations up to 8%, without any heat, by transferring the collagen from one syringe to another, back and forth several times in less than one minute. When reacted with oxidized starch, the irradiated collagen and starch crosslink to form a highly viscous foam.

### Ex Vivo Adhesion Test

Squares of defatted porcine dermis, 25 mm×25 mm, are glued together with the products (samples A, B, C), described below, to be tested. They are then incubated for 45 minutes at +37° C. The adhesion strength is measured by peeling off one piece of dermis from the other, by using a benchtop materials testing machine (Tinius Olsen, ECME 230). The peeling curves are analyzed with QMat-Pro software.

### Preparation of the Products

Sample A: Beta-irradiated esterified collagen—batch RGK00026—was solubilized at a concentration of 4% w/v. Two ml of the corresponding solution in a first syringe were mixed with 0.7 ml of 1.5% w/v oxidized starch—batch ZFE00523 x2—diluted in a second syringe, by transferring the products from one syringe to another one, back and forth, several times.

Sample B: Beta-irradiated esterified collagen—batch RGK00026—was also used alone, without oxidized starch at a concentration of 8% w/v.

Sample C: Prevadh™ foam was prepared from instructions for use from a SOFRADIM commercial kit. Two ml of heated collagen at a concentration of 16% w/v—batch SCC23330—were crosslinked with 0.7 ml of oxidized starch at a concentration of 3% w/v—batch SA024016.

### Adhesion Strength

The results of the adhesion strength are provided in Table 6 below. When compared to the commercial product Prevadh™ foam, esterified collagen cross-linked with oxidized starch to produce a glue product that worked quite well. But alone, without crosslinker, the adhesive properties of esterified collagen, were low.

The adhesiveness of esterified collagen crosslinked with oxidized starch was satisfactory as the adhesive strength reached 60% of the adhesive strength of Prevadh foam which obtains its adhesiveness with a much higher concentration of collagen (16% w/v) and crosslinker (3% w/v). Thus, the present collagen preparations having only 25% as much collagen and 50% crosslinker compared to the commercially available Prevadh Foam product provided 60% of the adhesive strength as the commercial product.

TABLE 6

| Sample | Tensile Adhesion Strength (N) |
|---|---|
| A: Esterified collagen (4%) + | 9.1 ± 4.8 (n = 8) [60%] |
| Crosslinker (1.5%) | |
| B: Esterified collagen (8%) + | 5.4 ± 7.3 (n = 8) [35%] |
| No crosslinker | |
| C: Prevadh Foam (16%) + | 15.5 ± 6.6 (n = 8) [100%] |
| Crosslinker (3%) | |

Esterified collagen, when compared to acid and neutral collagen, was the only one to be safely sterilized by beta-irradiation at doses lower than 25 KGy. Analytical results have shown that irradiated esterified properties were similar to the non-irradiated counterpart.

### Example 2

### Preparation of Collagen

Collagen was prepared as described above in Example 1 for esterified collagen.

### Sterilization of Collagen

Esterified collagen syringes were prepared as described above in Example 1, but were sterilized by gamma-irradiation at different doses ranging from 6 KGy to 30 KGy, either at room temperature or at a temperature below 0° C., in dry ice.

### Analysis of Gamma-Irradiated Collagen

Esterified collagen gave translucent solutions showing some viscosity, but increasing the dose of γ irradiation clearly decreased the viscosity, except for collagens irradiated at doses above 25 KGy which are presumed to be completely degraded, This was correlated with electrophoretic analysis showing more obvious signs of degradation with increasing gamma-irradiation dose.

Gamma irradiation was less effective than beta-irradiation in keeping initial properties of esterified collagen. Even, at low doses, around 10 KGy, esterified collagens were obviously altered when sterilized by gamma-irradiation. Gamma-irradiation for the preparation of sterile preparation of esterified collagen is less advantageous than beta-irradiation.

It will be understood that various modifications may be made to the embodiments disclosed herein. Therefore, the above description should not be construed as limiting, but merely as an exemplification of preferred embodiments. Those skilled in the art will envision other modifications within the scope and spirit of the present disclosure. Such modifications and variations are intended to come within the scope of the following claims.

1. A method comprising:
chemically modifying collagen to render the collagen soluble at physiological pH;
neutralizing chemically modified collagen to a physiological pH;
packaging the neutralized chemically modified collagen in sealed units; and
irradiating the packaged, neutralized chemically modified collagen with a dose of radiation equal to or lower than about 25 KGy.

2. The method of claim I, further comprising.
drying the neutralized chemically modified collagen before packaging.

3. The method of claim 2, further comprising:
storing the dried, neutralized chemically modified collagen at or below about 300 C before the irradiating step.

4. The method of claim 1, wherein irradiating is irradiating with beta-radiation.

5. The method of claim 4, wherein the irradiating with beta-radiation is at a dosage from about 6 KGy to about 25 KGy.

6. The method of claim 4, wherein the irradiating with beta-radiation is at a dosage lower than about 15 KGy.

7. The method of claim 1, wherein irradiating is performed as multiple cycles of irradiating doses wherein the total dose of radiation is equal to or lower than about 25 KGy.

8. The method of claim 1, wherein irradiating is performed at a temperature below 0° C.

9. The method of claim 1, wherein chemically modifying collagen comprises esterifying collagen.

10. The method of claim 9, wherein esterifying collagen comprises reacting collagen with a water soluble aliphatic alcohol.

11. The method of claim 1, wherein packaging comprises sealing the neutralized chemically modified collagen in a syringe.

12. A collagen preparation comprising terminally sterilized neutralized chemically modified collagen that is soluble at physiological pH.

13. A collagen preparation as in claim 12, wherein the collagen has at least in part an intact triple helical structure.

14. A collagen preparation as in claim 12, wherein the collagen is porcine.

15. A fiber formed from a collagen preparation in accordance with claim 12.

16. A tissue sealant or tissue adhesive comprising a collagen preparation in accordance with claim 12.

17. The tissue sealant or tissue adhesive of claim 16, further including oxidized starch.

18. A composition comprising a collagen preparation as in claim 12 in combination with a bioactive agent.

19. A tissue filler comprising a collagen preparation in accordance with claim 12 that is injectable and contained within a syringe.

20. A bone filler comprising a collagen preparation in accordance with claim 12 comprising one or more bone morphogenic proteins, and wherein the bone filler is injectable and contained within a syringe.

21. A method comprising:
combining a collagen preparation comprising terminally sterilized neutralized chemically modified collagen that is soluble at physiological pH with a sterile composition comprising a bioactive agent to provide a formulation suitable for application to tissue.
